(19) United States
(12) Patent Application Publication
(10) Pub. No.: US 2007/0225268 A1
(43) Pub. Date: Sep. 27, 2007

(54) METHOD TO TREAT NEOPLASMS VIA GADOLINIUM STEREOTACTIC SYNCHROTRON RADIATION

(76) Inventors: Pupa Gilbert, Madison, WI (US); Minesh P. Mehta, Madison, WI (US); Francois Esteve, Grenoble cedex (FR)

Correspondence Address: Intellectual Property Department;DEWITT ROSS & STEVENS S.C., Excelsior Financial Centre, 8000 Excelsior Drive, Suite 401, Madison, WI, 53717-1914, US

(21) Appl. No.: 11/618,421

(22) Filed: Dec. 29, 2006

Related U.S. Application Data
(60) Provisional application No. 60/755,120, filed on Dec. 30, 2005.

(51) Int. Cl. A61K 31/555; A61K 31/28

(52) U.S. Cl. 514/185; 514/492

(57) ABSTRACT

Described is a method of inhibiting the growth of neoplastic cells. The method includes the steps of contacting neoplastic cells with a gadolinium-containing compound for a time sufficient to allow the gadolinium-containing compound to be internalized within the neoplastic cells. The neoplastic cells are then exposed to a photon flux sufficient to induce emission of Auger electrons from the gadolinium-containing compound. The emitted Auger electrons induce non-reparable double-stranded DNA cleavage within the neoplastic cells, which thus inhibits the growth of the neoplastic cells. The method is particularly suitable for treating glioblastomas in humans.

### CROSS-REFERENCE TO RELATED APPLICATIONS

Priority is hereby claimed to provisional application Ser. No. 60/755,120, filed Dec. 30, 2005, the entire contents of which are incorporated herein.

### FEDERAL FUNDING STATEMENT

This invention was made with United States government support awarded by the following agency: NSF 0084402. The United States has certain rights in this invention.

### INCORPORATION BY REFERENCE

Full bibliographic citations for the references cited herein are compiled in the bibliography section, immediately preceding the claims. The references cited in the bibliography are incorporated herein by reference.

### BACKGROUND

Glioblastoma multiforme, the most common primary intracranial malignancy in the United States, has an annual incidence of ˜12,000 cases; the incidence and mortality are equal, highlighting the uniformly fatal outcome of this disease and the need for new therapeutic approaches to treat it. The vast majority of glioblastoma multiforme patients succumb within 1 year of diagnosis. The conventional therapy consists of maximal resection of the diseased area, followed by postoperative radiotherapy to ˜60 Gy with concomitant temozolomide chemotherapy. The median survival time using this treatment route is just under 15 months (1). The brachytherapy and radiosurgery experience with this tumor suggests that the effective tumor-control dose is greater than 100 Gy, a radiation dose which cannot be attained in most patients without inordinate morbidity. Although molecular biology-based therapy and chemotherapy are the major focus of current clinical research efforts, a more immediate effect could potentially be achieved by developing radiotherapy methods that enhance dose delivery to cancer cells while sparing surrounding healthy tissues.

Recent experiments with iodinated contrast agents and synchrotron radiation show that it is feasible to enhance radiation lethality. This is due to the use of a synchrotron photon beam tunable to the optimal absorption energy (34 keV) for the iodinated contrast agent (2, 3). Platinum compounds have also been tested for synchrotron stereotactic radiotherapy (SSR) at 78.8 keV, resulting in a dramatic increase in DNA damage (4, 5). In vivo experiments in rats bearing F98 brain glioma showed the effectiveness of SSR using either iodine or platinum, both in stereotactic irradiation mode (6-8). In particular, outstanding results were obtained using intracranial infusion of cis-platinum one (1) day before exposure to 78.8 keV photons (15 Gy single dose delivery) with a 34% survival rate at one (1) year. To date, this is the best survival rate obtained in the F98 animal model (4). However, localization of the iodine and silver-containing sensitizing agents within the tumor cells far less than ideal, which limits the ultimate effectiveness of these treatments. Thus there remains a long-felt and unmet need for an effective treatment for glioblastoma multiforme and other types of non-responsive cancers.

### SUMMARY OF THE INVENTION

Synchrotron stereotactic radiotherapy with gadolinium (GdSSR), as described herein, is a novel approach to treating neoplasms in general and brain cancers in particular (especially gliomas). GdSSR comprises administering a tumor-specific, gadolinium-containing compound to a subject in need thereof, and then subsequently stereotactically irradiating the subject with X-rays, most preferably monochromatic X-rays having about 51 keV photon energy (such X-rays preferably being obtained from a synchrotron beamline). This energy (˜51 keV) is just above the gadolinium K-edge (i.e., just above the binding energy of the electrons in the K orbital of gadolinium) and therefore extracts electrons from the K-shell of gadolinium atoms via the photoelectric effect. The vacancy left behind by electron excitation and ejection is rapidly filled by fluorescence decay and by the Auger electron cascade from other shells. The emitted Auger electrons have high linear energy transfer and induce irreparable double-strand DNA breaks. However, the effective path length of the Auger electrons is extremely short; therefore, such double-stranded DNA breaks are maximized the gadolinium atoms are localized in close proximity to DNA (i.e., in or near the nucleus or the mitochondria of the neoplastic cells).

The advantage of GdSSR is the availability of gadolinium-containing compounds, such as motexafin gadolinium, that localize within the nuclei of cancerous cells, and which can be administered intravenously or intracranially. In previous studies, the present inventors have shown that Gd-diethylenetriaminepentaacetic acid (Gd-DTPA) penetrates the plasma membrane of glioblastoma multiforme cells, both in vitro and in vivo. These prior studies have also shown that Gd-DTPA accumulates to higher levels in the cell nucleus relative to the cytoplasm (in in vitro studies). These in vitro experiments were done on human glioblastomamultiforme cells in culture (TB10) exposed to Gd-DTPA obtained commercially (“MAGNEVIST”®-brand Gd-DTPA, a registered trademark of Berlex Laboratories). Three independent techniques, Microscope à Emission de Photoélectrons par Illumination Synchrotronique de Type Onduleur (an instrument built by the present inventors which provides 20-nm optimum resolution), inductively coupled plasma-mass spectrometry (ICP-MS), and time of flight secondary ion mass spectrometry (TOF-SIMS), all showed intracellular localization (9).

The present inventors also tested the in vivo biodistribution of Gd-DTPA in six glioblastoma multiforme patients injected with Gd-DTPA before tumor excision. The tumors were frozen and sectioned for ICP-MS and Microscope à Emission de Photoélectrons par Illumination Synchrotronique de Type Onduleur analysis. Evidence for Gd intracellular localization was found within the tumors in all six cases. However, the gadolinium localized in only 6.1% of the cell nuclei analyzed. Thus, while the data conclusively showed intracellular localization of Gd-DTPA, an observation not previously made, the data also revealed that Gd-DTPA is not an ideal X-ray sensitizer due to its limited cellular uptake.

However, those data did show (for the first time) that Gd injected in the blood stream in the form of Gd-DTPA not only reaches areas of blood-brain barrier breakdown, but also reaches glioblastoma multiforme cells, penetrates the cell membrane, and localizes within the nucleus (10). These observations were made possible because of the spatial resolution and chemical sensitivity of synchrotron spectromicroscopy, which enabled gadolinium concentrations to be evaluated at subcellular resolution, an approach not previously employed. The lack of such detailed analysis is likely the basis for the conventional wisdom that Gd-containing imaging agents do not penetrate intracellularly into glioblastoma multiforme.

To increase the effectiveness of the treatment, a gadolinium-containing agent having a greater likelihood of localization within the majority of tumor cells is needed. A new agent, motexafin gadolinium, is one such candidate that can be used in the present invention (11-14). (Note that Gd-DTPA will function within the present invention, but because it has low cellular uptake, it is not a preferred gadolinium-containing compound for use in the present method.) Motexafin gadolinium, a tripyrrolic pentadentate aromatic metallotexaphyrin, is a redox mediator that selectively reaches tumor cells and produces reactive oxygen species (15). It is sold commercially under the registered trademark “XCYTRIN®” (a registered trademark of Pharmacyclics, Inc., Sunnyvale, Calif.). Others have previously published data showing the tumor-specific and prolonged retention of motexafin gadolinium in brain metastatic cancer cells and glioblastoma multiforme cells (16,17). Prolonged and preferential accumulation and retention of motexafin gadolinium has also been shown in the phase I and II trials of this agent as a radiosensitizer for glioblastoma multiforme (18).

The fraction of cancer cells importing intracellular gadolinium into their nuclei is a variable that impacts the effectiveness of GdSSR for any given gadolinium-containing compound. Nuclear uptake is a function of the tumor specificity of various Gd-containing compounds. An assessment of this variable is therefore a prerequisite to the clinical testing of any gadolinium-containing compound for use in GdSSR. Using motexafin gadolinium, the Examples contained herein determined this localization variable in vitro. As described below, the intranuclear distribution of Gd in four human glioblastoma multiforme cell lines was evaluated after exposure to motexafin gadolinium. The results were reproduced and corroborated with three different microscopy techniques: X-ray photoelectron emission spectromicroscopy (X-PEEM), scanning transmission X-ray microscopy (STXM), and confocal fluorescence microscopy. Intranuclear localization was used as an end point because of the short path length of Auger electrons emitted by excited gadolinium atoms.

The best energy for GdSSR is calculated to be about 51 keV (6). Auger electrons emitted by gadolinium atoms, under X-ray illumination at 51 keV, range in energy from between 0 and about 41 keV with an average energy of about 7.63 keV (19). The average linear energy transfer is about 0.3 MeV/μm (10). The radiation length (or mean inelastic path) of Auger electrons is limited to less than about 150 nm (15 nm for the average energy of 7.63 keV; refs. 19 and 20). Due to this rather short path length of Auger electrons, it is much preferred for the gadolinium atoms to be localized in the immediate proximity of DNA (i.e., within or proximate to the nucleus and/or the mitochondria; refs. 9 and 10). When an Auger electron is emitted, it most likely will interact with water molecules within a radius of a few nanometers, producing hydroxyl radicals, which in turn locally propagate oxidative damage to DNA (21). Lethal double-stranded DNA breaks occur in proportion to the extent of the radiation-induced oxidative damage. Hence, determining how many cell nuclei in a tumor contain gadolinium is a fundamental parameter for gauging the efficacy of any given gadolinium-containing compound for use in the present invention.

Thus, a first version of the invention is directed to a method of inhibiting the growth of neoplastic cells. The method comprises a first step of contacting neoplastic cells with a gadolinium-containing compound for a time sufficient to allow the gadolinium-containing compound to be internalized within the neoplastic cells. Then, the neoplastic cells are exposed to a photon flux (preferably a flux of substantially monochromatic X-ray photons) sufficient to induce emission of Auger electrons from the gadolinium-containing compound. The emitted Auger electrons induce non-repairable double-stranded DNA cleavage within the neoplastic cells, which inhibits the growth of the neoplastic cells. In the preferred version of the invention, the neoplastic cells are contacted with gadolinium motexafin or a derivative thereof. The method is particularly effective when the neoplastic cells being treated are glioblastoma cells.

The method can be used to treat mammals, including humans.

Regardless of the neoplastic condition being treated, it is preferred that the neoplastic cells are contacted with the gadolinium-containing compound for a time sufficient to allow the gadolinium-containing compound to be internalized specifically within nuclei of the neoplastic cells. It is likewise preferred that the neoplastic cells are exposed to a photon flux of monochromatic X-rays having an energy of at least 51 keV.

### DESCRIPTION OF THE FIGURES

FIG. 1. Time dependence of Gd uptake in TB 10 (●), U87 (▪), T98G (▾), and M059K (▴) human glioblastoma multiforme cell lines. Two independent cell cultures per cell line were exposed to 100 μmol/L motexafin gadolinium (16 ppm Gd) for 0 to 72 hours, and the gadolinium concentration in cells was measured by ICP-MS.

FIGS. 2A and 2B. Confocal fluorescence micrographs of a TB10 cell exposed to 100 μmol/L motexafin gadolinium for 72 hours. FIG. 2A, motexafin gadolinium-associated fluorescence imaged using 488-nm excitation and a 522-nm barrier filter (as for FITC fluorescence). FIG. 2B, motexafin gadolinium-associated fluorescence imaged using 568-nm excitation and a 585-nm barrier filter (as for rhodamine fluorescence). Bar=20 μm.

FIGS. 3A, 3B, and 3C. Confocal microscopic images of motexafin gadolinium-related fluorescence from TB10 (FIG. 3A), T98G (FIG. 3B), and MO59K cells (FIG. 3C), exposed to 100 μmol/L motexafin gadolinium for 72 hours. All images are optical sections at the half-height of the cells. Bar=20 μm.

FIGS. 4A, 4B, 4C, and 4D. T₁-weighed MRIs obtained after a first dose of 4 mg/kg motexafin gadolinium (FIG. 4A) with no conventional contrast, after completing a 1-week, 5-dose loading course (FIG. 4B), after completion of all 13 doses, given over a total of 3 weeks (FIG. 4C) and 7 days after the 13th motexafin gadolinium injection (FIG. 4D). The tumor (glioblastoma multiforme) is clearly visualized on all four non-contrast, post-motexafin gadolinium images. Notice tumor specificity without normal brain parenchyma enhancement and the increase in tumor enhancement with increasing doses of motexafin gadolinium. Four external reference tubes, used for calibration, are also imaged. These contained 0.01, 0.05, 0.1, and 0.2 mg/mL motexafin gadolinium, respectively.

### DETAILED DESCRIPTION OF THE INVENTION

Abbreviations and Definitions: The following abbreviations and definitions are used through out the specification and claims. Terms not explicitly defined herein are to be given their accepted definitions in the fields of medicine and/or medical physics.

Derivative=When referring to a core compound, a derivative is compound that shares the structure of the core compound, but includes additional moieties affixed to the core compound. For example, an adjacent or near homolog is a derivative, as are hydroxylated and alkylated analogs of the core compound.

Gadolinium-containing compound=Any compound, complex, chelate, and the like, that contains at least one gadolinium atom, and which can be administered to mammalian subjects in amounts sufficient to act as X-ray sensitizing agents without inducing significant adverse side-effects in the subjects. Explicitly included in the definition of “gadolinium-containing compound” are motexafin gadolinium, Gd-DOTA, Gd-EDTA, and Gd-DTPA.

Gd-DOTA=gadolinium tetraazacyclododecanetetraacetic acid.

Gd-EDTA=gadolinium ethylenediaminetetraacetic acid.

Gd-DTPA=gadolinium diethylenetriaminepentaacetic acid.

GdSSR=gadolinium synchrotron stereotactic radiotherapy.

ICP-MS=inductively coupled plasma-mass spectrometry.

Motexafin gadolinium=A gadolinium-containing compound having the structure: 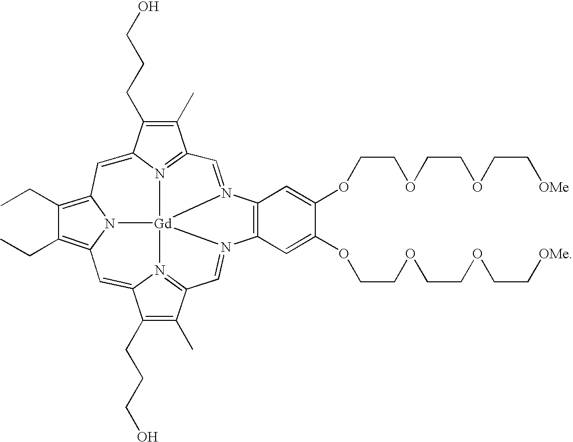
In the Literature, Motexafin Gadolinium is also Referred to as Gd-Tex.

SPHINX=Spectromicroscope for photoelectron imaging of nanostructures with X-rays (a spectromicroscope fabricated by Elmitec GmbH, Clausthal, Germany).

SSR=synchrotron stereotactic radiotherapy.

STXM=scanning transmission X-ray microscopy.

TOF-SIMS=time of flight secondary ion mass spectrometry.

X-PEEM=X-ray photoelectron emission spectromicroscopy

Gadolinium Synchrotron Stereotactic Radiotherapy:

Goorley and Nikjoo calculated and compared the theoretical effectiveness of three different cancer therapy approaches based on gadolinium: neutron capture, radioisotope decay, and photon activation therapy. Their results indicate that of the three cases, the photoelectric event has the highest Auger electron yield and the highest amount of energy deposited in a 10-μm sphere, making it more effective at killing the cell in which the reaction takes place (19). Tremendous improvement can be achieved if, as performed in the present invention, gadolinium atoms and X-ray-induced photoelectric events occur within the nuclei of cancer cells.

A fundamental parameter to establish is the minimum concentration of gadolinium that must be present in the nuclei of cancer cells in order to inhibit cancer cell growth via GdSSR. To address this question some calculations are necessary. A single gadolinium photo-absorption event and subsequent emission of Auger electrons close to DNA will kill a cell by inducing irreparable double-stranded DNA breaks. However, sufficient gadolinium must be present in the nuclei of a sufficient number of malignant cells in the tumor in order to inhibit growth of the tumor. The number of gadolinium photo-absorption events per cell nucleus (N) is obtained from the formula N=nΦμ, wherein n=number of Gd atoms per nucleus; Φ=photon fluence=10⁸ photons/mm² s×5 minutes=3×10¹⁰ photons/mm² (which is clinically acceptable) and corresponds to a dose of ˜20 Gy(4); and μ=absorption coefficient for gadolinium at 51 keV=4.5×10³ barn=4.5×10₋₁₉ mm². (For reference, the μ for carbon at the same energy is only 0.19 barn (34).) Additionally, a 51 keV photo-absorption from a K-shell electron occurs with 82% probability, and from an L-shell electron with 18% probability (19), thereby generating 8.05 Auger electrons, each having an average energy of 7.63 keV. The same photo-absorptions also generate the emission of two fluorescence photons, with average energy 34.9 keV (19). The Auger electrons, therefore, are the most abundant and also the most radio-biologically relevant particles because they have the highest linear energy transfer (0.3 MeV/μm) of the electrons emitted by each photo-absorption event. Using the above formula, one photo-absorption per nucleus can be achieved if 10⁸ Gd atoms/nucleus are present, which corresponds to a gadolinium concentration of about 10 ppm.

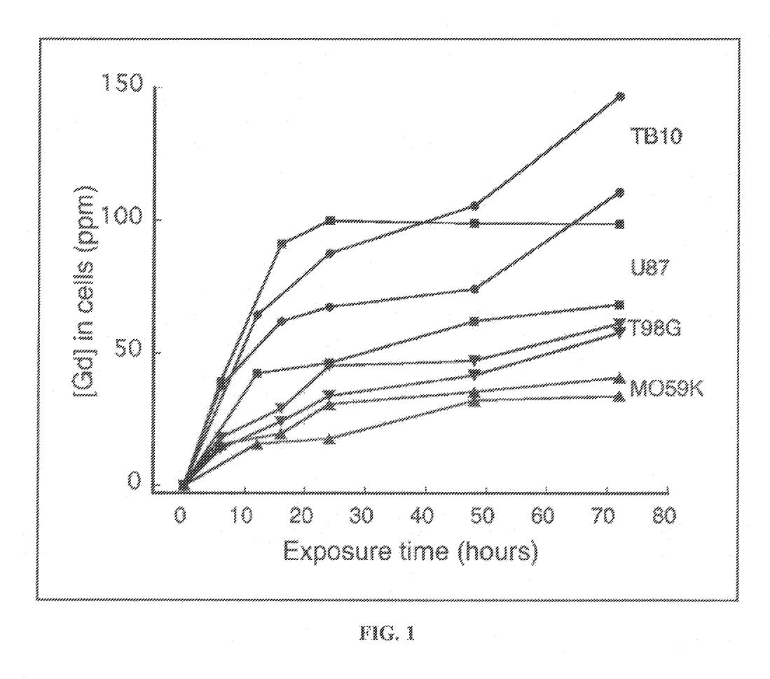

For GdSSR in vivo, assuming a Gaussian distribution of gadolinium molecules in cell nuclei as a result of uptake from the vasculature, Poisson statistics dictate that there must be an average of at least 24 gadolinium photo-absorption events per nucleus (i.e., a gadolinium concentration on the order of 100 ppm), to be sure that <1 in 10¹⁰ cells have zero photo-absorption events. That is, at an intranuclear gadolinium concentration of about 100 ppm, less than one cell per 10 billion cells will have zero photo-absorptions. This, of course, is the optimal outcome—that every cancerous cell within the tumor has at least one photo-absorption event with its consequent release of Auger electrons. This calculated gadolinium concentration in nuclei is comparable to the detection limit of spectromicroscopy, and is likewise comparable to the measured gadolinium concentrations reported in vitro (see FIG. 1) and in vivo (see Table 1).

The observation of a 70:1 tumor/normal tissue ratio almost two months after treatment (see the Examples) shows the tumor-specific uptake and prolonged retention of gadolinium within glioblastoma multiforme using motexafin gadolinium as a delivery vehicle. These data are particularly valuable as they show almost negligible gadolinium uptake in normal brain, an observation that is key to optimal treatment outcomes using GdSSR. Only a large differential in gadolinium concentration between tumor and normal brain yields optimal neoplastic cell death, while minimizing death or damage of normal cells. Significantly, even necrotic tumor shows 10- to 33-fold higher gadolinium concentration as compared to uninvolved brain areas (see Table 1).

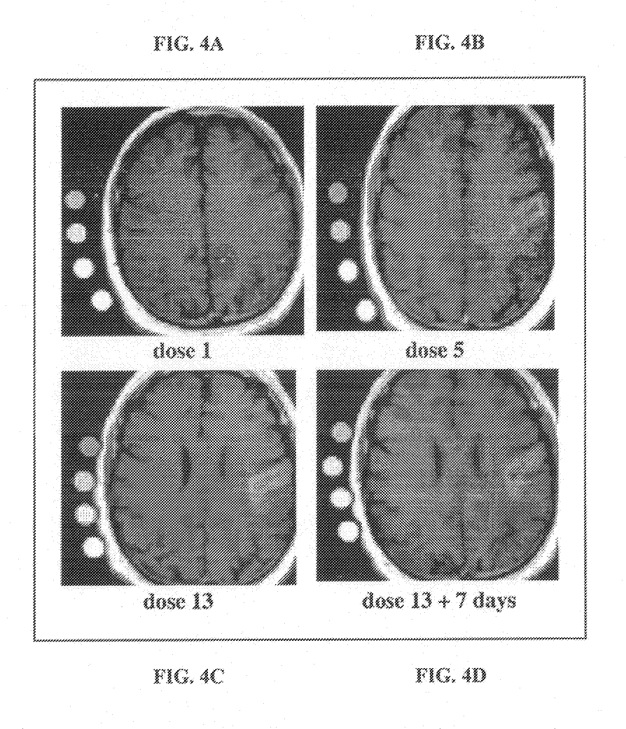

FIGS. 4A, 4B, 4C, and 4D show that much greater MRI enhancement in glioblastoma multiforme is achieved with repeated daily dosing with motexafin gadolinium than with a single dose. Thus, in the present invention, it is preferred that the repetitive doses of the gadolinium-containing compound be administered prior to exposing the patient to the X-rays.

If the concentration and/or distribution of gadolinium in tumor nuclei is less than ideal or if the gadolinium is inconsistently distributed across nuclei, tumor growth will be inhibited, but the tumor may not be destroyed entirely. Consequently, the larger the number of tumor nuclei a candidate gadolinium-containing compound reaches, the greater is the potential for GdSSR to inhibit the growth of neoplastic tumors (or to kill the tumor entirely).

The Examples presented herein evaluate the ability of motexafin gadolinium to localize intranuclearly in glioblastoma multiforme cells. Three different experimental approaches were used to observe motexafin gadolinium in the nuclei of glioblastoma multiforme cells. Using SPHINX, the percentage of nuclei containing gadolinium is 90% (n=180), with STXM it is 90% (n=200), whereas with confocal microscopy, it is 100% (n=481). STXM is more sensitive than SPHINX but did not result in an intranuclear gadolinium percentage greater than the corresponding percentage revealed by the SPHINX analysis. Confocal microscopy revealed 100% of the nuclei exhibiting motexafin gadolinium-related fluorescence. Fluorescence microscopy techniques cannot be used for most gadolinium-containing compounds because they are not fluorescent. Fluorescence detection of motexafin-gadolinium, however, is possible and quite sensitive—the most sensitive probe among the three used in the Examples.

Spectromicroscopic analysis, although slightly less sensitive than confocal fluorescence microscopy, is still necessary to provide definitive evidence of intranuclear gadolinium presence. This is because the fluorescence emission originates from the aromatic, expanded porphyrin component of the motexafin gadolinium molecule, and not from the gadolinium atom itself (32). It is conceivable that if the molecule is metabolized by the cell after uptake the texaphyrin is still present, but the Gd³⁺ ion is actively eliminated from the intracellular and/or intranuclear compartments. The motexafin gadolinium molecule was designed to be extremely stable in solution. In contrast to the gadolinium chelates (e.g., Gd-DTPA, Gd-DOTA, etc.) previously described in the literature, in motexafin gadolinium, the gadolinium is held within its texaphyrin macrocyclic core by coordinate covalent bonds (11, 12, 32). It is therefore unlikely that motexafin gadolinium would be denatured in solution. But it could be actively metabolized by the cell. (Thus, to be precise, the Examples refer to “motexafin gadolinium-related fluorescence,” and not “motexafin gadolinium fluorescence” when describing the confocal data.) It is possible that intracellularly, a motexafin gadolinium metabolite generates the detected fluorescence signal. Definitive proof of gadolinium presence, therefore, must be obtained with a direct elemental gadolinium probe. SPHINX and STXM analyses provide direct evidence and confirm intranuclear Gd presence in at least 90% of glioblastoma multiforme cells from four different cell lines exposed to motexafin gadolinium for 72 hours. See the Examples.

Administration of the gadolinium-containing compound is preferably via an intravenous or intracranial route. The formulations may be manufactured in unit dosage form and may be prepared by any of the methods well-known in the art of formulating pharmaceutically active ingredients. All methods include the step of bringing the gadolinium-containing compound into association with a carrier and one or more optional accessory ingredients. In general, the formulations are prepared by uniformly and intimately bringing the gadolinium-containing compound into association with a liquid carrier. The in vivo dosage in humans and other mammals depends largely upon the age, the general health, and the condition of the patient being treated. Determining the optimum dosage for any given mammal is essentially an empirical and ongoing process. Depending upon the stage of the tumor being treated, and the age of the patient, more (or less) aggressive treatment protocols may be required.

The gadolinium-containing compounds may be administered in the form of free acids or bases, or pharmaceutically suitable salts thereof. A pharmaceutically-suitable salt is any acid or base addition salt whose counter-ions are non-toxic to the patient in pharmaceutical doses of the salts, so that the beneficial inhibitory effects inherent in the free base or free acid are not vitiated by side-effects ascribable to the counter-ions. A host of pharmaceutically-suitable salts are well known in the art. For basic active ingredients, all acid addition salts are useful as sources of the free base form even if the particular salt, per se, is desired only as an intermediate product as, for example, when the salt is formed only for purposes of purification, and identification, or when it is used as intermediate in preparing a pharmaceutically-suitable salt by ion exchange procedures. Pharmaceutically-suitable salts include, without limitation, those derived from mineral acids and organic acids, explicitly including hydrohalides, e.g., hydrochlorides and hydrobromides, sulfates, phosphates, nitrates, sulfamates, acetates, citrates, lactates, tartrates, malonates, oxalates, salicylates, propionates, succinates, fumarates, maleates, methylene bis-b-hydroxynaphthoates, gentisates, isethionates, di-p-toluoyltartrates, methane sulfonates, ethanesulfonates, benzenesulfonates, p toluenesulfonates, cyclohexylsulfamates, quinates, and the like. Base addition salts include those derived from alkali or alkaline earth metal bases or conventional organic bases, such as triethylamine, pyridine, piperidine, morpholine, N-methylmorpholine, and the like.

The pharmaceutical formulation comprising the gadolinium-containing compound may further include one or more optional accessory ingredient(s) utilized in the art of pharmaceutical formulations, e.g., diluents, buffers, colorants, binders, surface-active agents, thickeners, lubricants, suspending agents, preservatives (including anti-oxidants) and the like.

The ultimate dosage of the gadolinium-containing compound administered to any given patient, as well as the ensuing dosage of X-ray photons, is at the discretion of the treating medical practitioner. Generally, however, one or more doses of the gadolinium-containing compound to yield an intranuclear gadolinium concentration within neoplastic cells of from about 10 to about 500 ppm is preferred, and a range of from about 50 to about 100 ppm still more preferred. The X-ray photon dose (administered in one or more discrete doses) is preferably from about 10 Gy to about 100 Gy, and more preferable still from about 10 Gy to about 50 Gy. The preferred energy of the X-ray photons should be at or slightly above the K-edge for gadolinium, i.e., about 51 keV.

### EXAMPLES

The following Examples are included solely to provide a more complete understanding of the invention described and claimed herein. The Examples do not limit the scope of the invention in any fashion.

Cell Culture and Exposure to Motexafin Gadolinium: Four well-established human glioblastoma multiforme cell lines were used for the in vitro studies: T98G (22), U87 (23), MO59K (24), and TB10 (9, 25). Cells were grown on precut 10 mm×10 mm silicon wafers (Silson, Northhampton, United Kingdom) for X-PEEM spectromicroscopy; on 1 mm×1 mm Si₃N₄ windows, 100 nm thick (Silson) for STXM spectromicroscopy; on tissue culture-treated polystyrene slides for confocal microscopy; and in Petri dishes for ICP-MS analysis. Subconfluent cultures were exposed to motexafin gadolinium (Pharmacyclics, Sunnyvale, Calif.), formulated at 2.3 mg/mL in 5% aqueous mannitol, and added to the culture medium to a final concentration of 100 μmol/L. Six different exposure times ranging from 0 to 72 hours were used for the ICP-MS time course measurements. For all microscopy experiments exposure times were kept constant at 72 hours. The cell appearance was not altered by motexafin gadolinium exposure.

During the culture and exposure periods, all cell lines were maintained in a humidified incubator at 37° C. and grown in DMEM/F12 medium containing 10% fetal bovine serum, 1% penicillin and streptomycin, and 1% nonessential amino acids (all from Mediatech, Herndon, Va.).

After exposure to motexafin gadolinium, the cell cultures on Si substrates for X-PEEM analysis were washed three times with PBS solution to remove free Gd, fixed in 4% paraformaldehyde in PBS for 20 minutes, double-washed in Milli-Q-water, and air dried. At this point, some cell cultures were sputtered with 3-kV argon ions at 10⁻⁵ Torr for 10 minutes; others were not. Sputtering removes the topmost portion of the cell to reveal the inner nuclear compartment of fixed cells (26). Sputtered and unsputtered cells were then ashed in UV/O₃ for 140 hours (27). Parallel cultures were run and treated identically for ICP-MS measurements of the bulk Gd concentration. Cell cultures for STXM analysis on Si₃N₄ windows, after motexafin gadolinium exposure, were washed, fixed, washed, and air-dried. Cell cultures for confocal microscopy were grown on a slide flask (Nunc, Rochester, N.Y.) containing a tissue culture-treated polystyrene slide. They were allowed to adhere for 24 hours and then exposed to 100 μmol/L motexafin gadolinium for 72 hours. The upper flask structure was then snapped away, and the cells were washed, fixed with 4% paraformaldehyde in PBS, washed, and air-dried. The slides were mounted with ProLong Antifade (Molecular Probes, Eugene, Oreg.) and imaged with confocal microscopy. Each sample was prepared in duplicate, triplicate, or quadruplicate.

ICP-MS Analysis: Cell cultures for ICP-MS bulk gadolinium concentration analysis, after exposure, were washed, digested in 1 mL of 1 N HNO₃, and scraped from the Petri dishes with a plastic spatula. The number of cells in each culture, typically 2 to 20×10⁵ per sample, was measured from a small aliquot. The total cell volume was then calculated, knowing the average volume of cells for each cell line. These were measured across six separate samples for each cell line, giving the following mean results: 62,400 TB10 cells/μL, 74,400 T98G cells/μL, 15,000 MO59K cells/μL, and 9,200 U87 cells/μL. Once the accurate cell volume in each motexafin gadolinium-exposed sample was obtained using these numbers, the ICP-MS results could be normalized (typical volume of cells, 10-40 μL/sample). This strategy gave reproducible results across two repeated cultures, with errors usually within 25% (see FIG. 1).

Tissue samples from three glioblastoma multiforme patients were also analyzed with ICP-MS (Table 1). These were collected from glioblastoma multiforme tissue and plasma for patients 1 and 2, and from tumor necrotic and uninvolved brain areas for the postmortem patient 3. All gadolinium concentrations were normalized to the measured tissue weight and volume.

X-PEEM Spectromicroscopy Analysis: X-PEEM spectromicroscopy analysis was done using the Spectromicroscope for Photoelectron Imaging of Nanostructures with X-rays (SPHINX) instrument (Elmitec GmbH, Clausthal, Germany), installed on the HERMON beamline at the Synchrotron Radiation Center (Madison, Wis.). SPHINX has an optimum lateral resolution of 10 nm (28) and acquires X-ray absorption spectra with a resolving power up to 15,000 (E/ΔE) in the 60 to 1,300 eV energy range. Phosphorus distribution maps were acquired to localize the nucleus in each cell. These were obtained by digital ratio of images at 139 and 132 eV, on-peak and pre-peak of the P2p edge (29). For trace concentration Gd analysis, Gd location maps were extracted as described previously (10, 28, 29) at the Gd3d edge (also known as M-edge) at ˜1,183 eV. In such Gd maps (data not shown), binned 4×4, the resolution is four times lower than in the images, and the Gd pixel size=1.4×1.4=2 μm². From the ICP-MS results of FIG. 1 and many other reference standards, this spectromicroscopy trace element analysis is estimated to have a minimum detection limit of <1,000 ppm. Cell and tissue ashing increases gadolinium concentration by a factor of 10; therefore, the detection limit was on the order of ≦100 ppm in all cells analyzed. In this Example, one Gd-containing pixel per cell nucleus was deemed sufficient to count that cell as one with a Gd-containing nucleus (29).

Ashing in UV/O₃ selectively removes carbon, a major element in cells and tissues, and consequently enhances the relative concentration of the other elements. Ashing takes place at ambient air pressure and temperature. Over the course of 100-200 hours, ashing slowly “flattens” the cell morphology. It is particularly useful when the element to be localized by spectromicroscopy is present in trace concentrations (27).

In the SPHINX experiments reported here, the cell nuclei were identified from the P distribution map in 180 TB 10 cells. The number of cell nuclei containing at least 1 Gd pixel were counted. The effectiveness of motexafin gadolinium in reaching glioblastoma multiforme nuclei was also evaluated.

STXM Spectromicroscopy Analysis: The STXM instrument on beamline 11.0.2 at the Advanced Light Source in Berkeley uses an undulator source and is optimized for the Gd3d absorption energy. This spectromicroscope (30,31) is both faster and more sensitive than X-PEEM for Gd detection. Ashing of the cell cultures for STXM analysis, therefore, was not required. Three cell cultures were analyzed, containing 34 T98G cells, 27 MO59K cells, and 139 TB10 cells, respectively. For Gd detection in cells, images were acquired on-peak and off-peak, at 1,183 and 1,178 eV. The ratio of these two images provided a Gd distribution map. Although this instrument does not allow the detection of phosphorus for identification of nuclei, cell nuclei seem thicker and denser in transmitted X-rays, and their outline can easily be visualized, allowing adequate scoring of Gd uptake.

Confocal Microscopy: Three of the four cell lines (TB10, MO59K, and T98G) were analyzed using a Bio-Rad (Richmond, Calif.) MRC 1024ES confocal microscope. This instrument can simultaneously measure the fluorescence from three channels, with excitation wavelengths from the three lines of a krypton/argon laser and emission through three independent barrier filters. These are 568 and 585 nm (rhodamine channel); 488 and 522 nm (FITC channel), and 488 and 680 nm (Cy 5 channel). The most intense fluorescence signal from motexafin gadolinium-treated cells was observed in the rhodamine channel; therefore, all images were recorded only with the 568 to 585 nm settings.

The aperture was set for minimal optical section thickness, and laser power and photodetector gain were set to image cell fluorescence within the 8-bit grayscale range of the photodetector. Five consecutive optical sections were obtained vertically through the cells to verify that motexafin gadolinium fluorescence originated from the nucleus and not only from the cytoplasm above or below the nucleus.

Magnetic Resonance Imaging: Magnetic resonance images (MRI) from one glioblastoma multiforme patient participating in a clinical trial of motexafin gadolinium and conventional radiotherapy at the University of California at Los Angeles (Protocol “A phase I dose escalating study of the safety and tolerability of gadolinium texaphyrin as a radiation sensitizer in patients with primary Glioblastoma Multiforme,” University of California at Los Angeles Institutional Review Board approval no. 97-09-042). The patient was injected with 4 mg/kg motexafin gadolinium five times per week, then thrice per week, up to 13 doses. T₁-weighed MRIs were obtained immediately after 1, 5, and 13 doses. We also collected images 7 days after the last dose, to prove the long-term, intracellular retention of this drug.

Results of the Examples:

In FIG. 1, the ICP-MS results obtained on the four human glioblastoma cell lines are presented in graphic form. These results show total uptake of gadolinium in cells, without regard to gadolinium location within the cell. The concentration of gadolinium after a 72-hour exposure to motexafin gadolinium varies slightly across the cell lines. Using the Student t test, gadolinium concentration in TB 10 cells was found to be significantly greater than in MO59K and T98G cells. Gadolinium concentration in T98G cells was also significantly greater than in MO59K cells. The molecular mechanistic explanation for this phenomenon is currently not known, but the unifying observation across the four cell lines is that they all take up motexafin gadolinium intracellularly and concentrate it with respect to the exposure solution. The 100 μmol/L motexafin gadolinium exposure solution contained 16 ppm Gd, whereas the concentration in the cells varied between ˜40 and ˜120 ppm after a 72-hour exposure to motexafin gadolinium. The intracellular gadolinium concentration, therefore, is 2.5 to 7.5 times greater than the extracellular gadolinium concentration.

An additional uniform observation across all four cell lines is that the concentration of gadolinium increases with duration of exposure to motexafin gadolinium and exceeds the exposure solution concentration after the first 6 hours. Although the intracellular gadolinium concentration continues to increase beyond 6 hours, the rate of increase diminishes after 12 hours, as shown by the decrease in slope for all four cell lines. FIG. 1 summarizes these results.

The absolute concentration of gadolinium in the cells, however, does not provide sufficient evidence that the gadolinium is present within the nuclei of the cells. The subcellular localization of gadolinium cannot be assessed by ICP-MS, but can be resolved by spectromicroscopic methods. Randomly selected TB10 cells (n=180) from three separate samples were analyzed with the SPHINX spectromicroscope. It was found that 90% of them (162) contained at least one gadolinium pixel in the nucleus (data not shown).

Interestingly, some gadolinium pixels appeared on the silicon substrate, where no cellular structures are visible. This result is peculiar to motexafin gadolinium and was not observed with other gadolinium-containing compounds (e.g., Gd-DTPA or Gd-1,4,7,10-tetra-azacylododecane-N,N[N′,N″,N′″-tetraacetic acid][Gd-DOTA]; see ref 10). Motexafin gadolinium was observed on the substrate with all three microscopies used in the Examples.

Of the 180 TB10 cells analyzed, 72 were sputtered and 108 were unsputtered. Of these, 63 and 99 cells, respectively, contained gadolinium. The change in the percentage of Gd-containing nuclei with sputtering, from 92% before sputtering to 88% after sputtering, is not significant, which indicates that the gadolinium detected in the nucleus was mostly intranuclear and not deposited on the cell surfaces and removed by sputtering. In all cell cultures, gadolinium pixels also appeared in the cytoplasm at higher densities than in the nuclei. It is possible that more cells contain gadolinium at a concentration below the SPHINX detection limit. Therefore other motexafin gadolinium-exposed cell cultures were analyzed with the more sensitive STXM spectromicroscope (data not shown).

In the STXM Gd maps, it was observed that gadolinium is distributed heterogeneously in the cells. It seems most intense in spots on the order of 1 μm in size, and these are denser around the nuclei than in the nuclei. These spots are very similar in size, density, and distribution to the gadolinium pixels observed with SPHINX. They are also denser around the nuclei than in the nuclei.

Overall, 34 randomly selected T98G cells, 27 MO59K cells, and 139 TB10 cells with STXM were analyzed. Thirty-one (90%) of the T98G cells, 26 of the MO59K (96%), and 122 of the TB10 cells (88%) contained gadolinium in their nuclei. Although ICP-MS had indicated that motexafin gadolinium uptake in TB10 cells is significantly greater than in T98G or MO59K cells, these Examples reveal that the percentage of nuclei taking up gadolinium is not significantly different between these cell lines (see the discussion regarding the confocal microscopy results, below). Overall, 90% of the cells analyzed with STXM (including T98G and TB10) had at least one gadolinium pixel in their nuclei. This result confirms and verifies with higher sensitivity the intranuclear presence of gadolinium already observed with SPHINX.

Although the results from the two spectromicroscopic techniques very strongly indicate intranuclear localization of gadolinium from motexafin gadolinium, there is one possible confounding variable. If gadolinium were present in high concentration in the cytoplasm and absent from the nuclei, the small portion of cytoplasm above and below the nucleus could be mistaken for the nucleus in the transmission STXM experiment. Similarly, in the SPHINX experiment, gadolinium atoms above the nucleus before ashing could seem to be localized in the nucleus after ashing and flattening of the cells. The sputtering of cells removes this as a source of error; the results on sputtered cells analyzed with SPHINX indicated that the gadolinium was truly located in the nuclei.

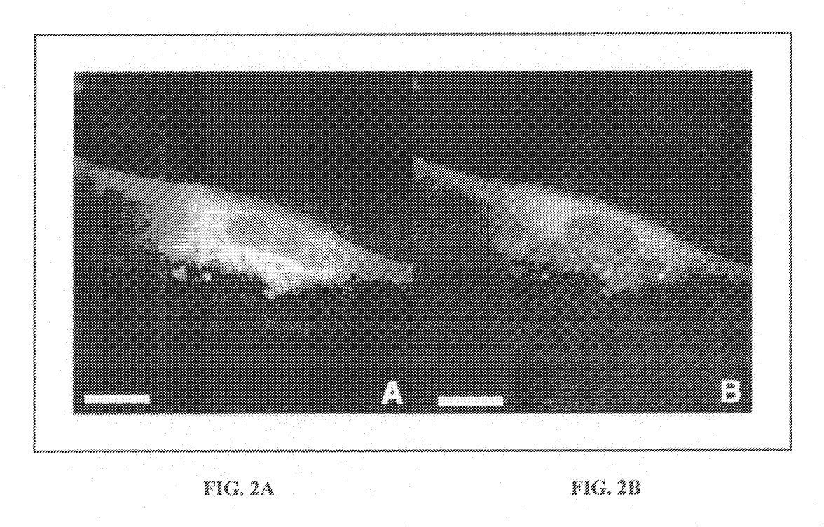

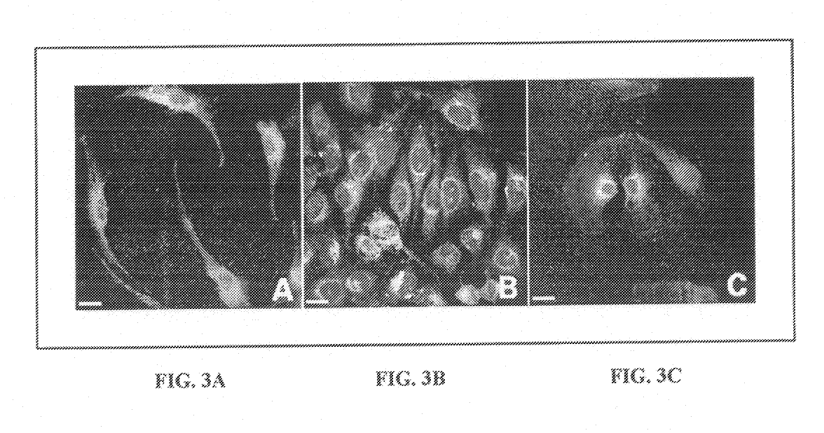

To validate intranuclear localization using techniques that require less manipulation of cells, motexafin gadolinium-exposed cells were analyzed with confocal microscopy. The motexafin gadolinium molecule is fluorescent due to the extended texaphyrin aromatic system surrounding the gadolinium atom (32). Motexafin gadolinium-treated cells emit fluorescence photons in both the FITC and the rhodamine spectral regions, as shown in FIGS. 2A and 2B. However, because the emission intensity in the rhodamine region is higher and does not interfere with autofluorescence, we analyzed all cells in this spectral range (See FIGS. 3A, 3B, and 3C). The motexafin gadolinium-related fluorescence intensity observed with confocal microscopy is always higher from the cytoplasm than from the nucleus. This result confirms the same observation previously made with SPHINX and STXM. All control cell cultures not exposed to motexafin gadolinium were imaged using the same wavelengths and showed no fluorescence or autofluorescence. In all motexafin gadolinium-exposed cells imaged with confocal fluorescence microscopy, as with SPHINX and STXM, higher intensities were observed from perinuclear spots, ≦1 μm in size, which could be interpreted as subcellular organelles preferentially taking up motexafin gadolinium, as suggested by Woodburn (32), or simply aggregates of motexafin gadolinium.

Most importantly, optical slicing across the cell thickness unequivocally shows that motexafin gadolinium fluorescence originates from within the cell nuclei. A total of 481 motexafin gadolinium-treated cells were analyzed with confocal microscopy: 100% of them exhibited motexafin gadolinium-related fluorescence in cytoplasm and nuclei. None of the 143 control cells revealed fluorescence.

Table 1 contains a compilation the raw data acquired by ICP-MS of gadolinium concentration in glioblastoma multiforme patients injected with a single dose of motexafin gadolinium at 10 mg/kg.

Patients 1 and 2 had minimal MRI enhancement with this single dose of motexafin gadolinium. Much greater MRI enhancement in glioblastoma multiforme is achieved with repeated daily dosing. The autopsy data on patient 3 indicate that the average tumor/normal tissue ratio is on average 70:1 (varying between 37 and 133), almost 2 months after treatment with multiple doses. This observation speaks to the tumor specific uptake and prolonged retention of Gd within glioblastoma multiforme cells using motexafin gadolinium as a delivery vehicle. Necrotic tumor shows 10- to 33-fold greater gadolinium concentration as compared to uninvolved normal brain. The significance of these results is manifest: motexafin gadolinium is selectively imported into glioblastoma cells as compared to normal cells, where it can then be selectively induced to omit Auger electrons to selectively inhibit the growth of those same glioblastoma cells.

TABLE 1
Intratumor (GBM) Gd measurements

| Patient 1 | | | | Patient 2 | | | | Patient 3 (autopsy) | |
|---|---|---|---|---|---|---|---|---|---|
| Time | | Source | [Gd] ppm | time | | Source | [Gd] ppm | Source | [Gd] ppm |
| Pre | | Plasma | 0.0003 | pre | | Plasma | 0.0021 | Tumor | 8.542 |
| 5 | min | Plasma | 12.973 | 5 | min | Plasma | 14.043 | Tumor | 8.022 |
| 45 | min | Plasma | 7.349 | 45 | min | Plasma | 8.143 | Necrosis within tumor | 2.144 |
| 2:30 | hrs | Plasma | 3.415 | 45 | min | Tumor | 22.589 | Uninvolved brain area | 0.216 |
| 2:30 | hrs | Tumor | 7.266 | 1:11 | hrs | Tumor | 29.728 | Uninvolved brain area | 0.126 |
| 3 | hrs | Plasma | 4.079 | 1:14 | hrs | Plasma | 6.536 | Uninvolved brain area | 0.068 |
| 3 | hrs | Tumor | 2.475 | 1:18 | hrs | Tumor | 19.580 | Uninvolved brain area | 0.143 |
| 24 | hrs | Plasma | 1.700 | 1:30 | hrs | Plasma | 6.309 | Uninvolved brain area | 0.109 |
| | | | | 3 | hrs | Plasma | 5.109 | Uninvolved brain area | 0.064 |
| | | | | 24 | hrs | Plasma | 1.770 | | |

NOTE:
Gd concentrations measured with ICP-MS. Patients 1 and 2 were injected with a single dose of Motexafin-Gd (10 mg/kg), before tumor resection. Patient 3 died 57 days after the last of multiple Motexafin-Gd doses, and the autopsy data are also reported.

Prolonged and preferential accumulation and retention of motexafin gadolinium in glioblastoma multiforme, as well as enhancement with repeated administration was also shown in the phase I and II trials of this agent as a radiosensitizer for glioblastoma multiforme (33). FIGS. 4A, 4B, 4C, and 4D together show enhancement in MRI and retention after multiple injections of motexafin gadolinium.

### BIBLIOGRAPHY

1. Stupp R, van den Bent M J, Hegi M E. Optimal role of temozolomide in the treatment of malignant gliomas. Curr Neurol Neurosci Rep 2005;5:198-206.
2. Estève F, Corde S, Elleaume H, Adam J F, et al. Enhanced radio sensitivity with iodinated contrast agents using monochromatic synchrotron X-rays on human cancerous cells. Acad Radiol 2002;9:S540-3.
3. Corde S, Joubert A, Adam J F, et al. Synchrotron radiation based experimental determination of the optimal energy for cell radiotoxicity enhancement following photoelectric effect on stable iodinated compounds. Br J Cancer 2004;91 :544-51.
4. Biston M C, Joubert A, Adam J F, et al. Cure of Fisher rats bearing radioresistant F98 glioma treated with cis-platinum and irradiated with monochromatic synchrotron X-rays. Cancer Res 2004;64:2317-23.
5. Corde S, Balosso J, Elleaume H, et al. Synchrotron photoactivation of cisplatin elicits an extra number of DNA breaks that stimulate RAD51-mediated repair pathways. Cancer Res 2003;63:3221-7.
6. Adam J F, Elleaume H, Joubert A, et al. Synchrotron radiation therapy of malignant brain glioma loaded with an iodinated contrast agent: first trial on rats bearing F98 gliomas. Int J Radiat Oncol Biol Phys 2003;57:1413-26.
7. Adam J F, Joubert A, Biston M C, et al. Enhanced delivery of iodine for synchrotron stereotactic radiosurgery by means of intracarotid injection and blood brain barrier disruption. Int J Rad Oncol Biol Phys 2005;61 :1173-82.
8. Adam J F, Joubert A, Biston M C, et al. Prolonged survival of Fischer rats bearing F98 glioma after iodine-enhanced synchrotron stereotactic radiotherapy. Int J Rad Oncol Biol Phys, in press 2005.
9. De Stasio G, Casalbore P, Pallini R, et al. Gadolinium in human glioblastoma cells for gadolinium neutron capture therapy. Cancer Res 2001 ;61:4272-7.
10. De Stasio G, Rajesh D, Casalbore P, et al. Are gadolinium contrast agents suitable for gadolinium neutron capture therapy? Neurol Res 2005;27:387-98.
11. Mody T D, Fu L, Sessler J L. Texaphyrins: synthesis and development of a novel class of therapeutic agents. In: Karlin K D, editor. Progress in inorganic chemistry. New York: John Wiley & Sons Inc; 2001. p. 551-98.
12. Sessler J L, Miller R A. Texaphyrins: new drugs with diverse clinical applications in radiation and photodynamic therapy. Biochem Pharmacol 2000;59:733-9.
13. Motexafin gadolinium: gadolinium (III) texaphyrin gadolinium texaphyrin Gd-Tex GdT2B2 PCI 0120. Drugs R D 2004;5:52-7.
14. Perez O D, Nolan G P, Magda D, et al. Motexafin gadolinium (Gd-Tex) selectively induces apoptosis in HIV-1 infected CD4⁺ T helper cells. Proc Natl Acad Sci USA 2002;99:2270-4.
15. Magda D, Lepp C, Gerasimchuk N, et al. Redox cycling by motexafin gadolinium enhances cellular response to ionizing radiation by forming reactive oxygen species. Int J Radiat Oncol Biol Phys 2001;51:1025-36.
16. Mehta M P, Shapiro W R, Glantz M J, et al. Lead-in phase to randomized trial of motexafin gadolinium and whole-brain radiation for patients with brain metastases: centralized assessment of magnetic resonance imaging neurocognitive and neurologic end points. J Clin Oncol 2002;20:3445-53.
17. Young S W, Qing F, Harriman A. Gadolinium(III) texaphyrin: a tumor selective radiation sensitizer that is detectable by MRI. Proc Natl Acad Sci USA 1996;93:6610-5.
18. Wu G N, Ford J M, Alger J R. The radiation enhancer motexafin gadolinium (MGd) does not penetrate the blood brain barrier in glioblastoma multiforme patients undergoing post-operative fractionated radiation therapy. Int J Radiat Oncol Biol Phys 2003;57:S329-30.
19. Goorley T, Nikjoo H. Electron and photon spectra for three gadolinium-based cancer therapy approaches. Radiat Res 2000;154:556-63.
20. Powell C J, Jablonski A. NIST electron inelastic-mean-free-path database, version 1.1. Gaithersburg (MD): National Institute of Standards and Technology; 2000.
21. Hall E J. In: Radiobiology for the radiologist. 5th ed. Philadelphia: Lippincott Williams & Wilkins; 2000. p. 19-20.
22. Stein G H. T98G: an anchorage-independent human tumor cell line that exhibits stationary phase G1 arrest in vitro. J Cell Physiol 1979;99:43-54.
23. Haas-Kogan D A, Kogan S S, Yount G, et al. p53 function influences the effect of fractionated radiotherapy on glioblastoma tumors. Int J Radiat Oncol Biol Phys 1999;43:399-403.
24. Wang J, Hu L, Allalunis-Turner M J, et al. Radiation-induced damage in two human glioma cell lines as measured by the nucleoid assay. Anticancer Res 1997;17:4615-8.
25. Falchetti M L, Pierconti F, Casalbore P, et al. Glioblastoma induces vascular endothelial cells to express telomerase in vitro. Cancer Res 2003;63:3750-4.
26. De Stasio G, Frazer B H, Girasole M, et al. Imaging the cell surface: argon sputtering to expose inner cell structures. Microsc Res Tech 2004;63:115-21.
27. Gilbert B, Perfetti L, Hansen R, et al. UV-ozone ashing of cells and tissues for spatially resolved trace element analysis. Front Biosci 2000;5:10-7.
28. Frazer B H, Girasole M, Wiese L M, et al. Spectromicroscope for the PHotoelectron Imaging of Nanostructures with X-rays (SPHINX): performance in biology medicine and geology. Ultramicroscopy 2004;99:87-94.
29. Frazer B H, Sonderegger B R, Gilbert B, et al. Mapping of physiological and trace elements with X-PEEM. J Phys Fr IV 2003;104:349-52.
30. Kilcoyne A L D, Tyliszczak T, Steele W F, et al. Interferometer-controlled scanning transmission X-ray microscopes at the Advanced Light Source. J Synchrotron Radiat 2003;10:125-36.
31. Tyliszczak T, Warwick T, Kilcoyne A L D, et al. Soft X-ray scanning transmission microscope working in an extended energy range at the advanced light source. In: Synchrotron Radiation Instrumentation 2003, AIP Conference Proceedings. 2004;705: 1356-9.
32. Woodburn K W. Intracellular localization of the radiation enhancer motexafin gadolinium using interferometric Fourier fluorescence microscopy. J Pharmacol Exp Ther 2001;297:888-94.
33. Miles D R, Smith J A, Phan S C, et al. Population pharmacokinetics of motexafin gadolinium in adults with brain metastases or glioblastoma multiforme. J Clin Pharmacol 2005;45:299-312.
34. Hubbell J H. Photon cross sections attenuation coefficients, and energy absorption coefficients from 10 keV to 100 GeV. NSRDS-NBS 29. Gaithersburg (MD): National Bureau of Standards; 1969.

What is claimed is:

1. A method of inhibiting the growth of neoplastic cells, the method comprising:
(a) contacting neoplastic cells with a gadolinium-containing compound for a time sufficient to allow the gadolinium-containing compound to be internalized within the neoplastic cells; and then
(b) exposing the neoplastic cells from step (a) to a photon flux sufficient to induce emission of Auger electrons from the gadolinium-containing compound, wherein the emitted Auger electrons induce non-repairable double-stranded DNA cleavage within the neoplastic cells, whereby growth of the neoplastic cells is inhibited.

2. The method of claim 1, wherein in step (a), the neoplastic cells are contacted with gadolinium motexafin or a derivative thereof.

3. The method of claim 1, wherein in step (a), the neoplastic cells are glioblastoma cells.

4. The method of claim 1, wherein in step (a), the neoplastic cells are human glioma cells.

5. The method of claim 1, wherein in step (a), the neoplastic cells are mammalian glioma cells within a mammalian subject suffering from glioma, and the cells are contacted with the Gd-containing compound by administering the Gd-containing compound to the mammalian subject.

6. The method of claim 5, wherein the mammalian subject is a human.

7. The method of claim 1, wherein in step (a), the neoplastic cells are contacted with a gadolinium-containing compound for a time sufficient to allow the gadolinium-containing compound to be internalized specifically within nuclei of the neoplastic cells

8. The method of any one of claims 1, 2, 3, 4, 5, 6, or 7, wherein in step (b), the neoplastic cells are exposed to a photon flux of monochromatic X-rays having an energy of at least about 51 keV.

9. A method of treating glioma in mammalian subjects, comprising:
(a) to a subject suffering from glioma, administering, intravenously or intracranially, an amount of a non-toxic, Gd-containing compound, the amount being sufficient to allow the compound to be internalized specifically within glioma cells within the subject; and then
(b) exposing the glioma cells from step (a) to a photon flux sufficient to induce emission of Auger electrons from the gadolinium-containing compound, wherein the emitted Auger electrons induce non-repairable double-stranded DNA cleavage within the glioma cells, whereby growth of the glioma cells is inhibited.

10. The method of claim 9, wherein in step (a), gadolinium motexafin or a derivative thereof is administered to the subject.

11. The method of claim 9, wherein the mammalian subject is a human.

12. The method of claim 9, wherein in step (a), the amount of the gadolinium-containing compound administered is sufficient to allow the gadolinium-containing compound to be internalized specifically within nuclei of the glioma cells.

13. The method of any one of claims 9, 10, 11, or 12, wherein in step (b), the glioma cells are exposed to a photon flux of monochromatic X-rays having an energy of at least 51 keV.
